(19) United States
(12) Patent Application Publication
(10) Pub. No.: US 2026/0207515 A1
(43) Pub. Date: Jul. 23, 2026

(54) Taste Masking Composition and Process

(71) Applicant: Tulex Pharmaceuticals Inc., Cranbury, NJ (US)

(72) Inventors: Yu-Hsing Tu, West Windsor, NJ (US); Shanmuka Harish Chalamuri, Cranbury, NJ (US); Ashok Perumal, Monmouth Junction, NJ (US)

(21) Appl. No.: 19/453,937

(22) Filed: Jan. 20, 2026

Related U.S. Application Data
(60) Provisional application No. 63/746,980, filed on Jan. 18, 2025.

(51) Int. Cl. A61K 9/24; A61K 9/20; A61K 9/28

(52) U.S. Cl. CPC A61K 9/209; A61K 9/2013; A61K 9/2846; A61K 9/2866; A61K 9/2893

(57) ABSTRACT

The invention is directed to a taste-masked pharmaceutical composition dosage form that includes a core comprising a pharmaceutically active ingredient and one or more pharmaceutically acceptable excipients, a seal coating encasing the core and a taste masking coating encasing the seal coated core. The taste masking coating includes a polymer that is insoluble at a pH above 5 and soluble below a pH of 5, an optional plasticizer and an anti-tacking agent present in an amount of between 70-140% w/w of the polymer. In a process for forming the coated core, the taste masking coating is applied to the seal coated core at a spray rate of 2.3-3.6 grams/min/kg and heating is applied in the coating apparatus such that an exhaust temperature of 36-40° C. from the coating apparatus is attained.

### BACKGROUND OF THE INVENTION

Bitter tasting drugs in tablet and capsule form often require taste masking. When such drugs are orally administered, merely placing the dosage form in the mouth or swallowing the dosage form can result in the patient experiencing an unpleasant, bitter taste. As a result of the bitter taste the patient may avoid taking the drug which could result in non-compliance to the prescribed dosing regimen. To address the bitter taste, tablets, capsules, and pellets or granules filled into capsules typically are coated with a taste masking coating, but the coating may still allow some of the drug to leach out in the mouth or upon swallowing may result in a bitter taste due to gastric reflux. The coating can be applied to granules, pellets, tablets and capsules. Taste masking coatings typically include a polymer that covers the surface of the granules, pellets, tablets or capsules. Another approach is to include a sweetener in the dosage form to improve the drug's palatability. Use of a sweetener undesirably adds additional agents to the composition and, further, may not completely mask the bitterness of the drug. Examples of bitter drugs are numerous, well known in the art, and include Acetaminophen, Ibuprofen, Topiramate, Diazoxide, Metronidazole, Clarithromycin, Erythromycin, Tetracycline, Ciprofloxacin, Chloramphenicol, Quinine, Chloroquine, Primaquine, Diphenhydramine, Chlorpheniramine, Propranolol, Quinidine, Digoxin, Verapamil, Amitriptyline, Fluoxetine, Carbamazepine, Phenytoin, Albendazole, Mebendazole, Ivermectin, Ketoconazole, Griseofulvin, Theophylline.

### SUMMARY OF THE INVENTION

In one general aspect, the invention is directed to a taste-masked pharmaceutical composition dosage form comprising a core comprising a pharmaceutically active ingredient and one or more pharmaceutically acceptable excipients; a seal coating encasing the core; and a taste masking coating encasing the seal coated core. The taste masking coating comprises a polymer and one or more anti-tacking agents. The polymer is insoluble at a pH above 5 and soluble below a pH of 5. The one or more anti-tacking agents are present in an amount of between 70-140% w/w of the polymer.

Embodiments of the dosage form may comprise one or more of the following features. For example, in one embodiment the seal coating can be a water soluble seal coating. For example, the polymer is selected from one or more of N,N-dimethylaminoethyl methacrylate, methylmethacrylate, butylmethacrylate and a copolymer of methyl methacrylate (MMA) and diethylaminoethyl methacrylate. The methyl methacrylate (MMA) and diethylaminoethyl methacrylate (DEAEMA) copolymer may have a molar ratio of the monomers MMA and DEAEMA in the copolymer of about 7:3. The polymer may be N,N-dimethylaminoethyl methacrylate with methylmethacrylate and butylmethacrylate.

The anti-tacking agent may be selected from one or more of talc, colloidal silicon dioxide, magnesium stearate, calcium stearate, glyceryl behenate, glycerol monostearate, polyethylene glycol (PEG), microcrystalline cellulose, stearic acid, fumed silica, kaolin, magnesium trisilicate, powdered starch and tribasic calcium phosphate. The anti-tacking agent may be selected from one or more of talc, stearic acid and colloidal silicon dioxide. The anti-tacking agent may be present in an amount of between 70-100% w/w of the polymer. The anti-tacking agent may be present in an amount of between 100-120% w/w of the polymer.

The plasticizer may be selected from one or more of acetyltributyl citrate (ATBC), triethyl citrate (TEC), dibutyl sebacate (DBS), tributyl citrate (TBC), acetyltriethyl citrate (ATEC), glycerin, polyethylene glycol monomethyl ether, triacetin, polyethylene glycol, propylene glycol, and sorbitol sorbitan solution.

The water soluble polymer in the seal coating may be one or more of hydroxypropyl cellulose, methyl cellulose, hydroxypropyl methylcellulose, hydroxyethyl cellulose, polyethylene glycol, polyvinyl alcohol, polyvinyl pyrrolidine (PVP), polyvinyl pyrrolidone-vinyl acetate copolymer (Copovidone), and poloxamer.

The composition has about zero release (0%) of the drug from the composition within 15 minutes in 900 mL pH 6.8 media in USP dissolution apparatus II at 50 rpm.

In another general aspect, the invention is directed to a process for forming a coated core containing a bitter tasing drug and encased by a taste-masking coating. The process includes the steps of providing a core comprising a pharmaceutically active ingredient and one or more pharmaceutically acceptable excipients; applying a seal coating to the core and applying a taste masking coating to the seal coated core whereby the core is encased by the seal coating. The taste masking coating comprises a polymer and an anti-tacking agent. The polymer is insoluble at a pH above 5 and soluble below a pH of 5. The anti-tacking agent is present in an amount of between 70-140% w/w of the polymer. The taste masking coating is applied to the seal coated core at a spray rate of 2.3-3.6 grams/min/kg and heating is applied in the coating apparatus such that an exhaust temperature of 36-40° C. from the coating apparatus is attained.

Embodiments of the process may include one or more of the following features. For example, the polymer may be selected from one or more of N,N-dimethylaminoethyl methacrylate, methylmethacrylate, butylmethacrylate and a copolymer of methyl methacrylate (MMA) and diethylaminoethyl methacrylate. The methyl methacrylate (MMA) and diethylaminoethyl methacrylate (DEAEMA) copolymer may have a molar ratio of the monomers MMA and DEAEMA in the copolymer of about 7:3. The polymer may be N,N-dimethylaminoethyl methacrylate with methylmethacrylate and butylmethacrylate.

The anti-tacking agent may be selected from one or more of talc, colloidal silicon dioxide, magnesium stearate, calcium stearate, glyceryl behenate, glycerol monostearate, polyethylene glycol (PEG), microcrystalline cellulose, stearic acid, fumed silica, kaolin, magnesium trisilicate, powdered starch and tribasic calcium phosphate. The anti-tacking agent may be selected from one or more of stearic acid, talc and silicon dioxide. The anti-tacking agent may be present in an amount of between 70-100% w/w of the polymer. The anti-tacking agent may be present in an amount of between 100-120% w/w of the polymer.

The plasticizer may be selected from one or more of acetyltributyl citrate (ATBC), triethyl citrate (TEC), dibutyl sebacate (DBS), tributyl citrate (TBC), acetyltriethyl citrate (ATEC), glycerin, polyethylene glycol monomethyl ether, triacetin, polyethylene glycol, propylene glycol, and sorbitol sorbitan solution.

The water soluble polymer in the seal coating may be one or more of hydroxypropyl cellulose, methyl cellulose, hydroxypropyl methylcellulose, hydroxyethyl cellulose, polyethylene glycol, polyvinyl alcohol, polyvinyl pyrrolidine (PVP), polyvinyl pyrrolidone-vinyl acetate copolymer (Copovidone), and poloxamer.

The composition may have about zero release (0%) of the drug from the composition within 15 minutes in 900 mL pH 6.8 media in USP dissolution apparatus II at 50 rpm.

### DESCRIPTION OF THE INVENTION

In the process of applying a polymer coating to a tablet, capsule, granule or pellet, the product temperature should be kept below the glass transition temperature to ensure that the polymer forms an even, complete coating that avoids peeling and cracking and avoids formation of agglomerations during the coating process. If the coating peels or cracks, the bitter drug will be released from the dosage form in the mouth and upon swallowing. Typically, the glass transition temperature (Tg) varies based on the coating polymer selected. The glass transition temperature of the polymer is lowered by the addition of a plasticizer and other coating excipients. A higher Tg can avoid coating process issues regarding product temperature control, however the subject polymers the inventors selected for reverse enteric coating, have a Tg between 40-50° C., and is even lower with added plasticizer. A reverse enteric coating is defined herein to mean a polymer that is insoluble at a pH above 5 but soluble below a pH of 5. Preferably the polymer is soluble in the stomach (i.e., at the pH of the stomach) to permit release of the active ingredient in the dosage form in the stomach.

Typically, the core of the bitter drug is first sealed with a water soluble polymer and then coated with a taste masking polymer. Examples of water soluble polymers for the seal coating over the core include hydroxypropyl cellulose, methyl cellulose, hydroxypropyl methylcellulose, hydroxyethyl cellulose, polyethylene glycol, polyvinyl alcohol, polyvinyl pyrrolidine (PVP), polyvinyl pyrrolidone-vinyl acetate copolymer (Copovidone), and poloxamer,

According to the invention, the taste masking polymer is a reverse enteric polymer that is insoluble at pH above 5 but soluble below pH of 5. In this manner, the reverse enteric polymer will not dissolve in the saliva of the mouth, which has a pH of about 7, or in the throat upon swallowing. However, in the acidic environment of the stomach the reverse enteric polymer will dissolve and release the bitter tasting drug. By releasing the drug in this environment, the patient should not be subject to the bitter taste of the drug. Further, even if the patient has some reflux, the patient should not experience the bitter taste of the drug in the mouth.

The inventors have attempted to use coatings of reverse enteric polymers to taste mask bitter drugs but have found that conventional techniques and excipient quantities nonetheless permits some bitter taste to be noticeable in the mouth. For example, the inventors have experimented with reverse enteric polymers such as a terpolymer based on N,N-dimethylaminoethyl methacrylate with methylmethacrylate and butylmethacrylate (available as Eudragit® EPO from Evonik); and methyl methacrylate (MMA) and diethylaminoethyl methacrylate (DEAEMA) copolymer (available as Kollicoat Smartseal® from BASF). Using conventional, recommended excipient quantities and coating parameters there is release of the drug during in vitro dissolution of the dosage form.

To avoid a patient noticing a bitter taste from a dosage form containing a bitter tasting drug, the inventors determined that there should be zero release (0%) of the drug within 15 minutes in 900 mL pH 6.8 media in USP dissolution apparatus II at 50 rpm. The inventors were unable to achieve this result using conventional excipient quantities. It is believed that the use of conventional excipient quantities resulted in sticking during the tablet or pellet coating process. The sticking will result in a flawed coating that results in early drug release. The inventors found that by increasing the amount of anti-tacking agent beyond the conventional amounts recommended in the literature, and carefully controlling the coating temperature, tablet sticking was reduced and drug was not released in the test conditions set out above. Examples of anti-tacking agents include talc, colloidal silicon dioxide, magnesium stearate, calcium stearate, glyceryl behenate, glycerol monostearate, polyethylene glycol (PEG), microcrystalline cellulose, and stearic acid. In one embodiment, magnesium stearate, glycerol monostearate and stearic acid, in combination or individually, are preferred. In another embodiment, talc and colloidal silicon dioxide, in combination or individually, are preferred. In another embodiment, stearic acid, talc and colloidal silicon dioxide, in combination or individually, are preferred.

The quantity of the combined anti-tacking agent(s), whether a single anti-tacking agent or multiple anti-tacking agents are used, should be in the range of about 70-140% by weight of the coating polymer used in the coating. In one embodiment, the quantity of anti-tacking agent(s) should be in the range of about 85-135% by weight of the coating polymer used in the coating, in the range of about 90-130% by weight of the coating polymer used in the coating, in the range of about 95-125% by weight of the coating polymer used in the coating, in the range of about 100-120% by weight of the coating polymer used in the coating, in the range of about 105-115% by weight of the coating polymer used in the coating, and individual integer values within the broad range.

Provided below in Table 1 are a coating formulation in accordance with the product literature recommendations for Eudragit® EPO and three examples (Examples 1, 2 and 3) according to the invention of taste masking coatings based on using basic butylated methacrylate copolymer along with talc or silicon dioxide added in excess of the recommended amounts to reduce sticking during coating. The Eudragit® E PO product literature includes recommendations for including an emulsifier, a salt former and an anti-tacking agent. The amounts listed in Table 1 for Comparative Example 1 are from the product literature for Eudragit® E PO.

TABLE 1

| | Composition in % w/w | | | |
|---|---|---|---|---|
| | Comparative | Exam- | Exam- | Exam- |
| Materials | Example 1 | ple 1 | ple 2 | ple 3 |
| Basic butylated | 8.57 | 7.28 | 7.70 | 6.93 |
| methacrylate copolymer* | | | | |
| Sodium lauryl sulfate | 0.86 | 0.74 | 0.78 | 0.70 |
| Stearic acid | 1.29 | 1.16 | 1.23 | 1.11 |
| Talc | 4.28 | 4.97 | 3.60 | 3.24 |
| Colloidal silicon dioxide | — | 0.85 | 1.68 | 3.02 |
| Water | 85.0 | 85.00 | 85.00 | 85.00 |
| Total | 100.0 | 100.0 | 100.0 | 100.0 |
| Weight percent of anti- | 65% | 95.88 | 84.55 | 106.35 |
| tacking agent(s) relative | | | | |
| to coating polymer | | | | |

*Eudragit ® E PO is commercially available basic butylated methacrylate copolymer

In Comparative Example 1, the stearic acid and talc both provide anti-tacking properties to the coating. The combined amount by weight of stearic acid and talc in the coating of Comparative Example 1 is approximately 65% by weight of the polymer weight (i.e., 5.57/8.57).

In Examples 1-3, the stearic acid, talc and silicon dioxide provide anti-tacking properties to the respective coating. The amount of the combined stearic acid, talc and colloidal silicon dioxide relative to the amount of polymer in Examples 1-3 ranges by weight from 84.55 to 106.35%. Based on physical observation, all three experimental examples had complete taste masking coatings without visual flaws and therefore should achieve the required dissolution. Testing determined that the amount of anti-tacking agents in experimental Example 3 met the requirement of no release of active ingredient in 15 minutes in 900 mL pH 6.8 media in USP dissolution apparatus II at 50 rpm.

Table 2 reports the recommended process parameters from the Eudragit® E PO product literature for applying the coating using an Accela Cota 10 with a batch size of 8 kg. As can be seen from the parameters listed for Example 1 in Table 2, the coating was applied at a temperature above that recommended by the Eudragit® E PO product literature. In particular, the exhaust temperature, which is a measure of the temperature leaving the tablet coater, 36-40° C., which is a range above the recommended range of 29-33° C.

TABLE 2

| | Range | |
|---|---|---|
| | From vendor's | |
| Process Parameter | product literature | Example 3 |
| Equipment | Accela Cota 10 | Freund Vector HI- |
| | | Coater System |
| | | (LDCS-PRO) |
| Batch size (tablets, kg) | 8 | 9.5-10.3 |
| Inlet air temperature (° C.) | 35-55 | 44-50 |
| Exhaust temperature (° C.) | 29-33 | 36-40 |
| Spray rate (g/min/kg) | 2 | 2.3-3.6 |
| Drying air volume/Drying | 224-262 | 475-515 |
| air flow (cfm) | | |
| Final Drying at 40° C. (hrs) | 2 (circulating | 1 (Tablet |
| | air oven) | coater) |

As indicated in Table 2, the process according to the invention can facilitate operating the process at a faster rate than recommended in the product literature. For example, the inventors were able to increase the spray rate to 2.3-3.6 grams/min/kg from the spray rate of 2 grams/min/kg recommended in the product literature. Coating at a higher rate requires elevated process conditions including the coating temperature, which in this case could result in process difficulties such as sticking or agglomeration resulting in coating imperfections. The inventors found that by using an increased amount of anti-tacking agent in addition to process controls, they were able to apply a uniform and complete coating of the taste masking polymer under conditions contrary to the product literature.

The following proposed formulations for taste-masking bitter tasting drugs using the improved coatings are made according to various embodiments of the invention.

### Example 4

| | | |
|---|---|---|
| Ingredient | % w/w | mg/tablet |

Core Tablet

| | | |
|---|---|---|
| Topiramate | 16.76 | 25.00 |
| Hydroxypropyl cellulose | 3.35 | 5.00 |
| Lactose monohydrate | 23.47 | 35.00 |
| Microcrystalline cellulose | 42.91 | 64.00 |
| Magnesium stearate | 0.67 | 1.00 |

Seal Coating

| | | |
|---|---|---|
| Opadry | 4.36 | 6.50 |

Taste-masking coating

| | | |
|---|---|---|
| Basic Butylated Methacrylate Copolymer | 3.94 | 5.88 |
| Talc | 1.97 | 2.94 |
| Silicon Dioxide | 1.52 | 2.27 |
| Stearic acid | 0.59 | 0.88 |
| Sodium lauryl sulfate | 0.39 | 0.59 |
| FD&C blue No. 2 | 0.05 | 0.07 |
| Total | 100.0 | 149.1 |

1. Topiramate is screened through #20 mesh sieve to de-agglomerate and added to a V-blender.
2. Hydroxypropyl cellulose, lactose monohydrate and microcrystalline cellulose are screened through 20 mesh sieve to de-agglomerate and added to the V-blender.
3. The ingredients are blended for 10 minutes
4. Magnesium stearate is screened through a #40 mesh sieve to de-agglomerate and added to the V-blender, and blended for 5 minutes
5. The final blend is compressed into tablets using a Sejong MRC-36 station tablet press using plain round bi-concave tooling to acceptable hardness
6. A solution of Opadry is prepared in purified water
7. The Opadry solution is sprayed on to the tablets using a Freund Vector Hi-Coater as to apply a seal coating on the tablets to about 5% w/w and tablets are dried in the tablet coater to remove excess moisture
8. Sodium lauryl sulfate, stearic acid and basic butylated methacrylate copolymer are dispersed in succession in purified water using a high shear mixer until completely dispersed
9. Talc is then added and dispersed using the high shear mixer, followed by FD&C blue No. 2
10. The coating dispersion is sprayed on to the tablets using a Freund Vector Hi-Coater as to apply the taste-masking coating on the tablets to about 9.25% w/w and tablets are dried in the tablet coater for 1 hour at 40° C. to cure.

Example 4 has a combined amount by weight of stearic acid, talc and colloidal silicon dioxide in the coating of approximately 103.57% by weight of the polymer weight (i.e., 6.09/5.88).

### Example 5

| | | |
|---|---|---|
| Ingredient | % w/w | mg/tablet |

Core Tablet

| | | |
|---|---|---|
| Acetaminophen | 68.93 | 500.00 |
| Hydroxypropylmethyl cellulose | 0.99 | 7.20 |
| Microcrystalline cellulose | 20.68 | 150.00 |
| Magnesium stearate | 0.50 | 3.60 |

Seal Coating

| | | |
|---|---|---|
| Opadry | 2.28 | 16.52 |

Taste-masking coating

| | | |
|---|---|---|
| Basic Butylated Methacrylate Copolymer | 3.26 | 23.64 |
| Talc | 1.86 | 13.47 |
| Colloidal Silicon dioxide | 0.80 | 5.77 |
| Stearic acid | 0.33 | 2.36 |
| sodium lauryl sulfate | 0.39 | 2.84 |
| Total | 100.0 | 725.4 |

1. Acetaminophen is screened through #20 mesh sieve to de-agglomerate and added to a V-blender.
2. Hydroxypropylmethyl cellulose and microcrystalline cellulose are screened through a #20 mesh sieve to de-agglomerate and added to the V-blender.
3. The ingredients are blended for 10 minutes.
4. Magnesium stearate is screened through a #40 mesh sieve to de-agglomerate and added to the V-blender, and blended for 5 minutes.
5. The final blend is compressed into tablets using a Sejong MRC-36 station tablet press using plain round bi-concave tooling to acceptable hardness.
6. A solution of Opadry is prepared in purified water.
7. The Opadry solution is sprayed on to the tablets using a Freund Vector Hi-Coater as to apply a seal coating on the tablets to about 2.5% w/w and tablets are dried in the tablet coater to remove excess moisture.
8. Sodium lauryl sulfate, stearic acid and basic butylated methacrylate copolymer are dispersed in succession in purified water using a high shear mixer until completely dispersed.
9. Talc is then added and dispersed using the high shear mixer.
10. The coating dispersion is sprayed on to the tablets using a Freund Vector Hi-Coater as to apply the taste-masking coating on the tablets to about 7.1% w/w and tablets are dried in the tablet coater for 1 hour at 40° C. to cure.

Example 5 has a combined amount by weight of stearic acid, talc and colloidal silicon dioxide in the coating of approximately 91.37% by weight of the polymer weight (i.e., 21.60/23.64).

### Example 6

| | | |
|---|---|---|
| Ingredient | % w/w | mg/tablet |

Core Tablet

| | | |
|---|---|---|
| Diazoxide | 7.45 | 10.00 |
| Polyvinylpyrrolidone | 4.10 | 5.50 |
| Microcrystalline cellulose | 74.47 | 100.00 |
| Magnesium stearate | 0.57 | 0.77 |

Seal Coating

| | | |
|---|---|---|
| Opadry | 4.32 | 5.80 |

Taste-masking coating

| | | |
|---|---|---|
| Basic Butylated Methacrylate Copolymer | 4.49 | 6.03 |
| Talc | 1.30 | 1.75 |
| Colloidal Silicon dioxide | 2.18 | 2.93 |
| Stearic acid | 0.67 | 0.90 |
| sodium lauryl sulfate | 0.45 | 0.60 |
| Total | 100.0 | 134.3 |

1. Diazoxide is screened through #20 mesh sieve to de-agglomerate and added to a Freund Vector GMX-25 high shear granulator.
2. Microcrystalline cellulose is screened a through #20 mesh sieve to de-agglomerate and added to the high shear granulator.
3. The ingredients are mixed for 5 minutes.
4. The ingredients are granulated using a solution of polyvinylpyrrolidone in purified water.
5. The wet granules are milled using a Quadro Comil to de-lump and dried in a circulating air oven at 50° C. to a moisture content of less than 2%.
6. The granules are milled using Quadro Comil and loaded into a V-blender.
7. Magnesium stearate is screened through a #40 mesh sieve to de-agglomerate and added to the V-blender, and blended for 5 minutes.
8. The final blend is compressed into tablets using a Sejong MRC-36 station tablet press using plain round bi-concave tooling to acceptable hardness.
9. A solution of Opadry is prepared in purified water.
10. The Opadry solution is sprayed on to the tablets using a Freund Vector Hi-Coater as to apply a seal coating on the tablets to about 5% w/w and tablets are dried in the tablet coater to remove excess moisture.
11. Sodium lauryl sulfate, stearic acid and basic butylated methacrylate copolymer are dispersed in succession in purified water using a high shear mixer until completely dispersed.
12. Talc is then added and dispersed using the high shear mixer, followed by silicon dioxide and FD&C blue No. 2.
13. The coating dispersion is sprayed on to the tablets using a Freund Vector Hi-Coater as to apply the taste-masking coating on the tablets to about 10% w/w and tablets are dried in the tablet coater for 1 hour at 40° C. to cure.

Example 6 has a combined amount by weight of stearic acid, talc and colloidal silicon dioxide in the coating of approximately 92.53% by weight of the polymer weight (i.e., 5.58/6.03).

### Example 7

| | | |
|---|---|---|
| Ingredient | % w/w | mg/capsule |

Drug pellets

| | | |
|---|---|---|
| MCC spheres | 61.37 | 120.20 |
| Topiramate | 12.76 | 25.00 |
| Polyvinylpyrrolidone | 1.28 | 2.50 |
| Talc | 5.11 | 10.00 |

Seal Coating

| | | |
|---|---|---|
| Hydroxypropylmethyl cellulose | 3.86 | 7.57 |
| Talc | 2.58 | 5.05 |

Taste masking Coating

| | | |
|---|---|---|
| Basic Butylated Methacrylate Copolymer | 7.23 | 14.16 |
| Silicon Dioxide | 2.35 | 4.60 |
| Glyceryl monostearate | 1.81 | 3.54 |
| Stearic acid | 1.08 | 2.12 |
| sodium lauryl sulfate | 0.58 | 1.13 |
| Total | 100.0 | 195.9 |

1. Polyvinylpyrrolidone solution is prepared in purified water using an overhead mixer.
2. Topiramate is solubilized in the solution followed by dispersion of talc using the overhead mixer.
3. The drug dispersion is sprayed on to MCC spheres using a Freund vector VFC-LAB 3 fluid bed processor equipped with a Wurster coating assembly.
4. The pellets are unloaded and dried in a circulating air oven at 60° C. to remove any excess moisture.
5. A solution of hydroxypropylmethyl cellulose is prepared in purified water using an overhead mixer.
6. Talc is added to the solution and mixed using the high shear mixer to disperse.
7. The seal coating dispersion is sprayed on to drug pellets using the Freund vector VFC-LAB 3 fluid bed processor equipped with a Wurster coating assembly.
8. The pellets are unloaded and in a circulating air oven at 60° C. to remove any excess moisture.
9. Sodium lauryl sulfate, stearic acid and basic butylated methacrylate copolymer are dispersed in succession in purified water using a high shear mixer until completely dispersed.
10. Glyceryl monostearate is dispersed in purified water separately using a high shear mixer.
11. The glyceryl monostearate dispersion is added to the polymer dispersion while mixing using an overhead mixer until uniformly dispersed.
12. The taste masking polymer dispersion is sprayed on to the seal coated pellets using the Freund vector VFC-LAB 3 fluid bed processor equipped with a Wurster coating assembly.
13. The pellets are unloaded and cured in a circulating air oven at 40° C. for 2 hours.
14. The pellets are filled into hard gelatin capsules with a target fill weight equivalent to 25 mg of topiramate.

Example 7 has a combined amount by weight of glyceryl monostearate, stearic acid and colloidal silicon dioxide in the coating of approximately 72.46% by weight of the polymer weight (i.e., 10.26/14.16).

### Example 8

| | | |
|---|---|---|
| Ingredient | % w/w | mg/capsule |

Drug pellets

| | | |
|---|---|---|
| MCC spheres | 61.41 | 120.20 |
| MCC spheres | 61.36 | 60.00 |
| Diazoxide | 10.23 | 10.00 |
| Hydroxypropylmethyl cellulose | 1.23 | 1.20 |
| Polysorbate 80 | 1.18 | 1.15 |
| Talc | 5.11 | 5.00 |

Seal Coating

| | | |
|---|---|---|
| Polyvinyl alcohol | 3.16 | 3.09 |
| Polyethylene glycol | 0.79 | 0.77 |

Taste masking Coating

| | | |
|---|---|---|
| Basic Butylated Methacrylate Copolymer | 6.93 | 6.78 |
| Talc | 4.57 | 4.47 |
| Silicon Dioxide | 3.99 | 3.90 |
| Stearic acid | 0.69 | 0.68 |
| Sodium lauryl sulfate | 0.76 | 0.75 |
| Total | 100.0 | 97.8 |

1. Hydroxypropylmethyl cellulose solution is prepared in purified water using an overhead mixer, followed by polysorbate 80.
2. Diazoxide is then dispersed in the above solution using a high shear mixer until completely dispersed.
3. Talc is then dispersed in the drug dispersion using the overhead mixer.
4. The drug dispersion is sprayed on to MCC spheres using a Freund vector VFC-LAB 3 fluid bed processor equipped with a Wurster coating assembly.
5. The pellets are unloaded and dried in a circulating air oven at 60° C. to remove any excess moisture.
6. A solution of polyvinyl alcohol and polyethylene glycol is prepared is prepared in purified water using an overhead mixer.
7. The seal coating dispersion is sprayed on to drug pellets using the Freund vector VFC-LAB 3 fluid bed processor equipped with a Wurster coating assembly.
8. The pellets are unloaded and in a circulating air oven at 60° C. to remove any excess moisture.
9. Sodium lauryl sulfate, stearic acid and basic butylated methacrylate copolymer are dispersed in succession in purified water using a high shear mixer until completely dispersed.
10. Talc is then added and dispersed using the high shear mixer, followed by silicon dioxide.
11. The taste masking polymer dispersion is sprayed on to the seal coated pellets using the Freund vector VFC-LAB 3 fluid bed processor equipped with a Wurster coating assembly.
12. The pellets are unloaded and cured in a circulating air oven at 40° C. for 2 hours.
13. The pellets are filled into hard gelatin capsules with a target fill weight equivalent to 10 mg of diazoxide.

Example 8 has a combined amount by weight of talc, stearic acid and colloidal silicon dioxide in the coating of approximately 133.48% by weight of the polymer weight (i.e., 9.05/6.78).

What is claimed:

1. A taste-masked pharmaceutical composition dosage form comprising:
a core comprising a pharmaceutically active ingredient and one or more pharmaceutically acceptable excipients;
a seal coating encasing the core; and
a taste masking coating encasing the seal coating,
wherein the taste masking coating comprises a polymer that is insoluble at a pH above 5 and soluble below a pH of 5 and an anti-tacking agent present in an amount of between 70-140% w/w of the polymer.

2. The taste-masked pharmaceutical composition dosage form of claim 1, wherein the polymer is selected from one or more of N,N-dimethylaminoethyl methacrylate, methylmethacrylate, butylmethacrylate and a copolymer of methyl methacrylate (MMA) and diethylaminoethyl methacrylate.

3. The taste-masked pharmaceutical composition dosage form of claim 2, wherein the methyl methacrylate (MMA) and diethylaminoethyl methacrylate (DEAEMA) copolymer has a molar ratio of the monomers MMA and DEAEMA in the copolymer of about 7:3.

4. The taste-masked pharmaceutical composition dosage form of claim 1, wherein the polymer is N,N-dimethylaminoethyl methacrylate with methylmethacrylate and butylmethacrylate.

5. The taste-masked pharmaceutical composition dosage form of claim 1, wherein the anti-tacking agent is selected from one or more of talc, colloidal silicon dioxide, magnesium stearate, calcium stearate, glyceryl behenate, glycerol monostearate, polyethylene glycol (PEG), microcrystalline cellulose, stearic acid, fumed silica, kaolin, magnesium trisilicate, powdered starch and tribasic calcium phosphate.

6. The taste-masked pharmaceutical composition dosage form of claim 1, wherein the anti-tacking agent is selected from one or more of talc, stearic acid and colloidal silicon dioxide.

7. The taste-masked pharmaceutical composition dosage form of claim 1, wherein the anti-tacking agent is present in an amount of between 70-100% w/w of the polymer.

8. The taste-masked pharmaceutical composition dosage form of claim 1, wherein the anti-tacking agent is present in an amount of between 100-120% w/w of the polymer.

9. The taste-masked pharmaceutical composition dosage form of claim 1, further comprising a plasticizer selected from one or more of acetyltributyl citrate (ATBC), triethyl citrate (TEC), dibutyl sebacate (DBS), tributyl citrate (TBC), acetyltriethyl citrate (ATEC), glycerin, polyethylene glycol monomethyl ether, triacetin, polyethylene glycol, propylene glycol, and sorbitol sorbitan solution.

10. The taste-masked pharmaceutical composition dosage form of claim 1, wherein the composition has about zero release (0%) of the drug within 15 minutes in 900 mL pH 6.8 media in USP dissolution apparatus II at 50 rpm.

11. A process for forming a coated core encased by a taste-masking coating, the process comprising:
providing a core comprising a pharmaceutically active ingredient and one or more pharmaceutically acceptable excipients;
coating the core with a seal coating; and
applying a taste masking coating to the seal coating whereby the seal coating is encased by the taste masking coating,
wherein the taste masking coating comprises a polymer that is insoluble at a pH above 5 and soluble below a pH of 5 and an anti-tacking agent present in an amount of between 70-140% w/w of the polymer,
wherein the taste masking coating is applied to the seal coated core at a spray rate of 2.3-3.6 grams/min/kg and heating is applied in the coating apparatus such that an exhaust temperature of 36-40° C. from the coating apparatus is attained.

12. The process of claim 11, wherein the polymer is selected from one or more of N,N-dimethylaminoethyl methacrylate, methylmethacrylate, butylmethacrylate and a copolymer of methyl methacrylate (MMA) and diethylaminoethyl methacrylate.

13. The process of claim 12, wherein the methyl methacrylate (MMA) and diethylaminoethyl methacrylate (DEAEMA) copolymer has a molar ratio of the monomers MMA and DEAEMA in the copolymer of about 7:3.

14. The process of claim 11, wherein the polymer is N,N-dimethylaminoethyl methacrylate with methylmethacrylate and butylmethacrylate.

15. The process of claim 11, wherein the anti-tacking agent is selected from one or more of talc, colloidal silicon dioxide, magnesium stearate, calcium stearate, glyceryl behenate, glycerol monostearate, polyethylene glycol (PEG), microcrystalline cellulose, stearic acid, fumed silica, kaolin, magnesium trisilicate, powdered starch and tribasic calcium phosphate.

16. The process of claim 15, wherein the anti-tacking agent is selected from one or more of stearic acid, talc and silicon dioxide.

17. The process of claim 11, wherein the anti-tacking agent is present in an amount of between 70-100% w/w of the polymer.

18. The process of claim 11, wherein the anti-tacking agent is present in an amount of between 100-120% w/w of the polymer.

19. The process of claim 11, further comprising a plasticizer selected from one or more of acetyltributyl citrate (ATBC), triethyl citrate (TEC), dibutyl sebacate (DBS), tributyl citrate (TBC), acetyltriethyl citrate (ATEC), glycerin, polyethylene glycol monomethyl ether, triacetin, polyethylene glycol, propylene glycol, and sorbitol sorbitan solution.

20. The process of claim 11, wherein the composition has about zero release (0%) of the drug within 15 minutes in 900 mL pH 6.8 media in USP dissolution apparatus II at 50 rpm.
